(19) United States
(12) Patent Application Publication
(10) Pub. No.: US 2026/0357183 A1
(43) Pub. Date: Nov. 12, 2026

(54) HIGH THROUGHPUT, FEEDBACK-CONTROLLED ELECTROPORATION MICRODEVICE FOR EFFICIENT MOLECULAR DELIVERY INTO SINGLE CELLS

(71) Applicant: Rutgers, The State University of New Jersey, New Brunswick, NJ (US)

(72) Inventors: Maria Atzampou, Attiki (GR); Hoa Lin, Belle Mead, NJ (US); David I. Shreiber, Whitehouse Station, NJ (US); Jerry W. Shan, Bridgewater, NJ (US); Jeffrey D. Zahn, Piscataway, NJ (US)

(73) Assignee: Rutgers, The State University of New Jersey, New Brunswick, NJ (US)

(21) Appl. No.: 19/118,528

(22) PCT Filed: Oct. 6, 2023
(86) PCT No.: PCT/US2023/076268

§ 371 Date: Apr. 4, 2025

Related U.S. Application Data
(60) Provisional application No. 63/378,770, filed on Oct. 7, 2022.

(51) Int. Cl. C12M 1/42; C12M 3/06; C12N 13/00

(52) U.S. Cl. CPC C12M 35/02; C12M 23/16; C12N 13/00

(57) ABSTRACT

Systems and methods for electroporating a biological cell are disclosed. The system includes a microfluidic channel adapted to receive a flow of a plurality of biological cells in a buffer solution. The microfluidic channel comprises a detection region and a pulsing region. The system also includes a first pair of electrodes adapted to apply a first electrical field across the detection region, and a second pair of electrodes adapted to apply a second electric field across the pulsing region.

### CROSS REFERENCE TO RELATED APPLICATIONS

This application claims priority to U.S. Application No. 63/378,770, filed Oct. 7, 2022, which is incorporated herein by reference in its entirety.

### STATEMENT REGARDING FEDERALLY SPONSORED RESEARCH

This invention was made with government support under Contract No. 1353918 awarded by the National Science Foundation. The government has certain rights in this invention.

### FIELD OF THE DISCLOSURE

The present disclosure relates to the field of cell electroporation and molecular delivery in general, using an intelligent, feedback-controlled, microscale electroporation system for transfecting single cells in a continuous-flow fashion.

### BACKGROUND OF THE INVENTION

Delivery of small and macromolecules—including, for example, DNA, RNA, drug molecules, imaging agents, peptides, antibodies, and enzymes—into cells is critical to realizing their full potential in a range of research and therapeutic applications; yet, intracellular delivery and transfection remain difficult tasks. Successful transfection is a rate-limiting step in many types of biomedical research and bioproduction workflows that govern markets including biopharmaceuticals, RNA interference screening, and stem cell research. However, this potential has not been realized, largely because of the difficulties in safely, effectively, and efficiently transfecting the cells. The challenges include variable and poor transfection efficiency, especially with hard-to-transfect cell lines such as primary cell lines and stem cell lines that are of significant interest for studies of developmental dynamics, drug discovery, and regenerative medicine.

During electroporation, genes or other macromolecules are mixed with the live cells in a buffer medium and short pulses of high electric field intensities are applied. The cell membranes are transiently permeabilized and the genes or macromolecules enter the cells. However, electroporation frequently falls short of the desired efficiency and reliability because of inadequate permeabilization and/or cell death due to over permeabilization.

Hence, there exists a need for a new and improved microfluidic electroporation device that can address the above challenges.

### SUMMARY OF THE INVENTION

In one aspect, the present disclosure provides a system for electroporating a biological cell, the system comprising a microfluidic channel adapted to receive a flow of a plurality of biological cells in a buffer solution, wherein the microfluidic channel comprises a detection region and a pulsing region, a first pair of electrodes adapted to apply a first electrical field across the detection region, and a second pair of electrodes adapted to apply a second electric field across the pulsing region.

Optionally, a second detection region may be included in the system downstream of the pulsing region. A controller unit may be adapted to control parameters of the second electric field based on a first impedance detected in the detection region and a second impedance detected in the second detection region.

In various embodiments, a third pair of electrodes may be included in the system downstream of the second pair of electrodes and adapted to apply a third electric field across the pulsing region. Optionally, the second electric field has a strength that is higher than that of the third electric field. Additionally and/or alternatively, the second electric field has a shorter duration than the third electric field. The second electric field may be configured to cause permeabilization of a cell. The third electric field may be configured to cause delivery of a molecule within the cell. The second pair of electrodes or the third pair of electrodes comprise interdigitated electrodes.

In one or more embodiments, the first electrical field is configured to detect presence of a cell between the first pair of electrodes. Optionally, the second electrical field is configured to electroporate the cell between the second pair of electrodes.

The system may also include a plurality of inlets configured to hydrodynamically focus the plurality of biological cells in a single cell flow within the microfluidic channel.

### BRIEF DESCRIPTION OF THE DRAWINGS

Embodiments will be described with reference to the following drawing figures, in which like numerals represent like items throughout the figures, and in which:

FIG. 1A illustrates an example electroporation system;

FIG. 1B illustrates an example electroporation system including two pulsing regions;

FIG. 1C illustrates an example electroporation system with interdigitated electrodes;

FIGS. 2A and 2B illustrate the effect of increasing the pulse duration on electro-transfection Efficiency (eTE) and survival rate (SR).

FIGS. 3A and 3B illustrate the effect of increasing the number of pulses on eTE and survival rate (SR).

FIGS. 4A and 4B illustrate the change in impedance before and after a cell is permeabilized, according to an embodiment;

FIGS. 5A and 5B show separation of permeabilization signal and delivery signal;

FIG. 6A illustrates changes in eTE upon application two signals in the pulsing region;

FIGS. 6B-6E illustrates Green Fluorescent Protein (GFP) expression in HEK293 cells following electroporation;

FIG. 7 is a flowchart illustrating an example electroporation method; and

FIG. 8 is a block diagram that is useful for understanding exemplary computer hardware which is capable of implementing the methods described herein.

### DETAILED DESCRIPTION OF THE INVENTION

Methods and systems are disclosed for cell electroporation and molecular delivery in using an intelligent, feedback controlled, microscale electroporation system for electroporating and transfecting flowing single cells.

It will be readily understood that the components of the embodiments as generally described herein and illustrated in the appended figures could be arranged and designed in a wide variety of different configurations. Thus, the following more detailed description of various embodiments, as represented in the figures, is not intended to limit the scope of the present disclosure, but is merely representative of various embodiments. While the various aspects of the embodiments are presented in drawings, the drawings are not necessarily drawn to scale unless specifically indicated.

The present disclosure may be embodied in other specific forms without departing from its spirit or essential characteristics. The described embodiments are to be considered in all respects as illustrative. The scope of the disclosure is, therefore, indicated by the appended claims.

Reference throughout this specification to features, advantages, or similar language does not imply that all of the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Thus, discussions of the features and advantages, and similar language, throughout the specification may, but do not necessarily, refer to the same embodiment.

Furthermore, the described features, advantages and characteristics of the disclosure may be combined in any suitable manner in one or more embodiments. One skilled in the relevant art will recognize, in light of the description herein, that the disclosure can be practiced without one or more of the specific features or advantages of a particular embodiment. In other instances, additional features and advantages may be recognized in certain embodiments that may not be present in all embodiments of the disclosure.

Reference throughout this specification to “one embodiment,” “an embodiment,” or similar language means that a particular feature, structure, or characteristic described in connection with the indicated embodiment is included in at least one embodiment of the present disclosure. Thus, the phrases “in one embodiment”, “in an embodiment”, and similar language throughout this specification may, but do not necessarily, all refer to the same embodiment.

As used in this document, the singular form “a”, “an”, and “the” include plural references unless the context clearly dictates otherwise. Unless defined otherwise, all technical and scientific terms used herein have the same meanings as commonly understood by one of ordinary skill in the art. As used in this document, the term “comprising” means “including, but not limited to”.

Electroporation is a means to access the cytoplasm of a cell for delivery of molecules. In the technique, an electric field, which can be applied in vitro or in vivo, transiently permeabilizes the cell membrane through which biologically active molecules can enter the cell, such as DNA, RNA, and amino acids. The current disclosure describes a system and method for a flow-based, automated cell detection-and-electroporation signal system to detect and electroporate cells.

Electroporation approaches can be divided into two categories: macroscale and microscale electroporation. Even though macroscale electroporation can process large number of cells per unit time, high levels of Joule heating from resistive dissipation though the solution medium interfere with the cell viability and recovery. Current microscale methods generally have low throughput. The reason why these microscale electroporation devices have low throughputs is mostly because they combine cell perfusion through microchannels with cell electroporation and monitoring. Other designs require static or trapped cells, which limit the number of treated cells per unit time. Flow-through devices can achieve larger number of treated cells per unit time, but they usually lack the ability to monitor the electroporation events.

For example, U.S. Pat. No. 10,927,333 (the disclosure of which is incorporated herein by reference) describes a microscale continuous-flow single-cell electroporation device capable of performing cell detection, pulse application, and real-time cell membrane permeabilization measurement. Specifically, the '333 Patent describes a device for continuously monitoring the current flowing across a detection area to determine the cell membrane impedance of a single cell flowing through a microfluidic channel before, during and after electroporation. The system further includes a sensing unit that may comprise a cell membrane permeabilization feedback control loop that monitors the impedance of a detection area of the microfluidic channel using a first electrical detection signal to determine when a single cell enters into the detection area. The single cell entering the detection area may be represented as a rise in impedance over a baseline threshold, resembling a pulse resistive sensor in, for example, a coulter counter. The application of a second electrical permeabilization signal permeabilizes the cell membrane of the single cell. The system may automatically adjust parameters of the second permeabilization signal until a predefined sub-lethal permeabilization threshold is met. The impedance of the detected cell is continuously monitored to determine when the sub-lethal permeabilization threshold is achieved. A third electrical delivery signal is applied to deliver molecules into the cell cytoplasm. The parameters of the third delivery signal may be automatically adjusted by the system. During this process, the impedance of the detection area and detected single cell may be continuously monitored to detect if the impedance change exceeds a predefined maximal threshold, which can cause possible irreversible cell damage, whereupon the feedback control loop turns off the third electric delivery signal and resets the system for the next passing cell.

The disclosed device in the '333 Patent has a limited throughput of about 2-4 cells/second because it utilizes the same electrode set for both sensing of the cells and permeabilization (i.e., pulsing) that causes a lag when switching between functions. The procedure as currently practiced cannot, therefore, be adapted for higher throughput required for real-world clinical applications. The current disclosure describes a device that includes at least two sets of electrodes, where a first set of electrodes is configured to sense the presence of a cell between the electrodes while a second set of electrodes is configured to permeabilize the cells passing between (i.e., separate the sensing region and the electropulsing region to create spatial control of permeabilization) in order to achieve high electroporation efficiency and intracellular delivery while avoiding solution electrolysis. Providing electrode set(s) specific for detection and pulsing (as described herein) eliminates the need for a restrictive cell-transit time between the electrodes that was determined by the sensing-triggering-pulsing time duration in the prior art devices that forced the cells to have a relatively low speed. The throughput of the disclosed methods and systems may be about 200-1000 cells/second. The disclosed device further prevents electrolysis of the solution by selection of an appropriate pulse parameters (e.g., shape, duration, and magnitude). The disclosure further describes novel electrode designs for preventing solution electrolysis during electroporation.

Optionally, a third set of electrodes may be used to monitor cell permeabilization after electroporation. In other words, impedance cytometers may be developed and integrated upstream and downstream of the permeabilization in order to monitor individual cell impedance changes following electroporation. Monitoring these changes for various pulsing conditions on a cell population level allow the system to identify the optimal pulse features for a particular cell type's required permeabilization threshold, and thus optimize the permeabilization for each individual cell type.

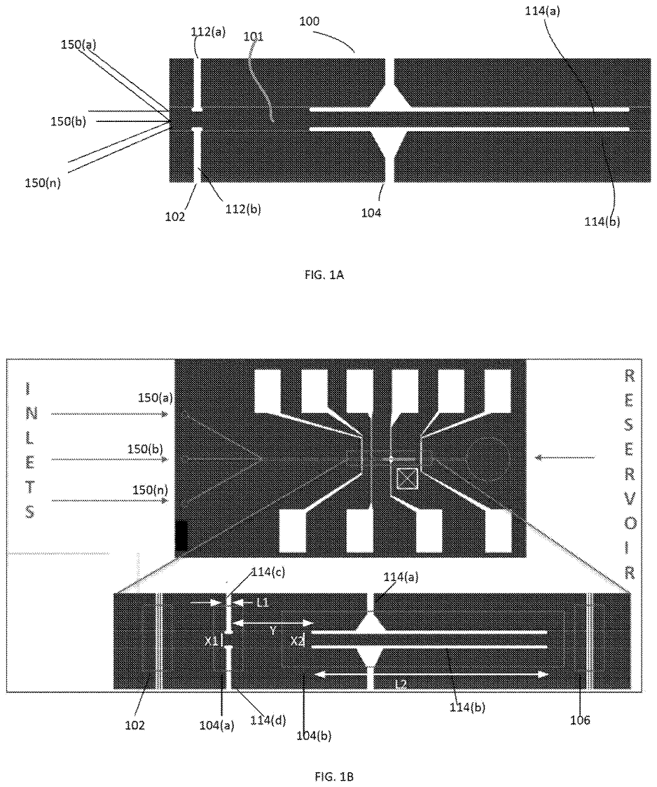

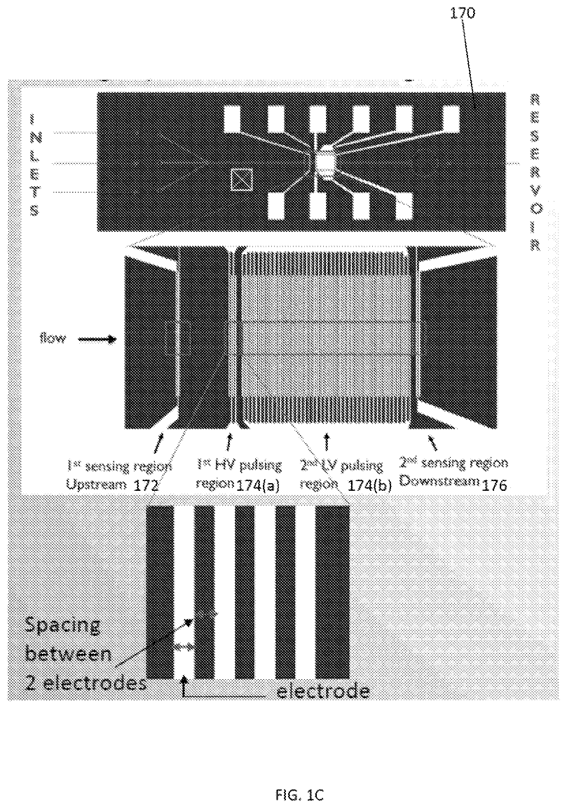

FIG. 1A illustrates an example device 100 for performing electroporation that includes microfluidic channel 101 including a first sensing region 102 configured for detecting the presence of a cell in the microfluidic channel 101, and a pulsing region 104 for electroporation of the cell passing through the microfluidic channel. The first sensing region 102 may be formed by using a first pair of electrodes 112(a) and 112(b) configured for application of a first electrical signal across the microfluidic channel 101. The pulsing sensing region 104 may be formed using a second pair of electrodes 114(a) and 114(b) configured or application of a second electrical signal across the microfluidic channel 101. As used herein, a signal is any time-varying waveform and may include, for example, an alternating current (AC) waveform or a direct current (DC) waveform. In some embodiments, a signal of the present invention may be an AC sinewave waveform, a single DC pulse waveform, or a series of DC pulse waveforms. The first and second set of electrodes may be kept in a continuously active mode to generate an appropriate signal at a desired voltage so as to improve the throughput of cells (since no cycling between different signal/voltages is required due to the continuous activation).

The device 100 may be designed and fabricated using microfabrication techniques known to those skilled in the art. For example, the microfluidic channel 101 (and/or the electrodes) may be patterned on glass slides using techniques such as lithography (e.g., photolithography). For example, the microfluidic channels are fabricated using standard soft lithography, where photolithography is used to pattern a negative photoresist on silicon wafers to act as polydimethylsiloxane (PDMS) master molds for replica molding of the microchannels and Ti/Pt electrodes on glass substrates. A PDMS solution is poured over the master mold and baked at 60 °C to produce a hardened negative relief. The PDMS is peeled from the master molds to create the microfluidic channels, and holes are punched for inlets and outlets. The PDMS and sputtered glass slide are treated with oxygen plasma to activate the surfaces and bonded together with feature alignment. In example embodiments, the microchannels may be 6 mm long, 280 μm wide, and 45 μm deep. The inlets are connected to a syringe pump (Harvard Apparatus, Cambridge, Mass.) using polyethylene tubing (Small Parts, Miami Lakes, Fla.) for cell and sample introduction. The inlets, tubing, and syringe pump parameters (e.g., dimensions, positioning, and pressure) may be selected to control the flow rate of cells through the microfluidic channel. Other fabrication techniques are within the scope of this disclosure.

In an embodiment, electrodes are fabricated using liftoff techniques on clean glass (e.g., silicon dioxide) slides. In an example embodiment, the electrodes are patterned using a lithographically defined positive photoresist masking layer (EVG620 Exposure system) to define the electrode areas, followed by sputtering a 1000 Å thick Ti/Pt layer (Kurt J. Lesker PVD75) and photoresist removal in acetone solution. Wires may be soldered on special connective pads patterned on the glass substrates to allow connection with external electronics. Alignment marks may be designed and added in order to be able to align the microfluidic channel in parallel with the electrode sets. Methanol can be used as a lubricant between the PDMS and the glass surfaces to help with the alignment.

The microfluidic channel 101 may be adapted to receive a flow of a plurality of biological cells in a buffer solution from a plurality of inlets 150(a)-(n). The microfluidic channel 101 may be designed to hydrodynamically-focus single cells for delivery between the electrodes in the pulsing region 104 via the sensing region 102. Specifically, the inlets are designed to accommodate hydrodynamic flow focusing of the cells in the middle of the channel in order for the cells to flow in a single file line. Hydrodynamically focusing the cells into single-file using fluid streams (e.g., a sheathing buffer) from side inlets 150(a)-(n) permits continuous cell introduction, and is preferred over a narrow single channel because of the propensity for those channels to clog. Cell concentrations and flow rates may be controlled to provide optimal spacing between cells for control of the electroporation parameters (as discussed below). Optionally, interdigitated electrodes (discussed below) that span across the microchannel may obviate the need for accurately positioning the cells between the electrodes and hydrodynamic focusing.

In the sensing region 102, the first pair of electrodes 112(a) and 112(b) may be configured to monitor the impedance of the sensing region 102 of the microfluidic channel using a first electrical detection signal to determine when a single cell enters into the sensing region 102. In an embodiment, the first electrical detection signal is an AC sine waveform of low amplitude. The single cell entering the detection area may be represented as a rise in impedance over a baseline threshold, resembling a pulse resistive sensor in, for example, a coulter counter. In some embodiments, the first electrical detection signal may be an AC waveform. In other embodiments, the first electrical detection signal may be a series of DC pulse waveforms.

In the pulsing region 104, the second pair of electrodes 114(a) and 114(b) may be configured to apply an electroporation signal across the pulsing region. In some embodiments, the electroporation signal may be a DC pulse waveform, a series of DC pulse waveforms (e.g., long DC pulses, a pulse train of high frequency DC pulses, etc.), square waves, an AC pulse waveform (e.g., sine waves), or combinations thereof.

Optionally, the electroporation signal may be dynamically tailored depending upon the type of cell detected within the sensing region (the type determined based on, for example, the amount of rise in impedance over a baseline threshold). Specifically, the electroporation signal may be chosen based on experimental data received from using the electroporation system on immobilized cells (for a particular cell type). For example, appropriate threshold levels, AC waveform parameters, and DC pulse train parameters may be determined and stored in a datastore (e.g., a database) for particular types of cells that are trapped in a microfluidic device with a small cell trap area (<5×5×10 μm³) within a pair of electrodes. Upon detecting a particular cell type, the system may use the cell type to retrieve the corresponding electric pulse parameters previously stored in the database. In some example embodiments, the electroporation signal may be selected or selectively tuned (to determine an optimal frequency and voltage) to cause electroporation of detected cells while preventing cell death and/or electrolysis of the cell suspension solution. The success of electroporation may be measured using factors such as, without limitation, a electrotransfection efficiency (eTE), cell viability, and start time of electrolysis. In some examples, as pulse intensity is increased (i.e., higher electric field strengths are applied), the pulse duration is shortened to maintain cell viability.

For example, when a square wave pulse is applied at a duty cycle of about 20% and a constant frequency of about 1 KHz, eTE of about 1% was observed between about 21-25 V of voltage without electrolysis (electrolysis was observed at voltage=25V) as shown in Table 1 below. When a square wave pulse is applied at a duty cycle of about 20% and a constant frequency of about 10 KHz, eTE of about 1% was observed at a voltage of about 25-28 V, which gradually increased with an increase in voltage till about 40V (electrolysis was observed at voltage=40V) as shown in Table 2 below.

TABLE 1
(Frequency = 1 Hz)

| HV (V) | 5 | 7 | 10 | 13 | 15 | 18 | 21 | 25 |
|---|---|---|---|---|---|---|---|---|
| eTE(%) | 0 | 0 | 0 | 0 | 0 | 0 | 1 | 1 |
| | | | | | | | (No electrolysis) | (electrolysis) |

TABLE 2
(Frequency = 10 Hz)

| HV (V) | 25 | 27.5 | 30 | 35 | 40 |
|---|---|---|---|---|---|
| eTE(%) | 1 | 1 | 3 | 10 | 13 (electrolysis) |

The highest eTE was observed when 113, 30V-square wave pulses were applied at a duty cycle of about 20% and a constant frequency of 10 KHz. This eTE was determined to be 76.44±8.52%, the cell viability was preserved and no electrolysis bubbles were formed.

In various embodiments, electric field strengths of about 0.1 to about 4 kV/cm, about 0.2 to about 3.5 kV/cm, about 0.3 to about 3 kV/cm, about 0.4 to about 2.5 kV/cm, about 0.5-2.0 kV/cm, about 0.6 to about 1.5 kV/cm, or the like may be applied to achieve electroporation without causing cell death and/or solution electrolysis. For such embodiments, the pulse durations may be about 1 μs to about 5 ms (typically, a 1 kV/cm pulse intensity may be achieved with a 1 ms DC pulse duration).

In some other embodiments, electroporation may be achieved using higher electric field strength pulses applied for a shorter duration in order to further avoid solution electrolysis. For example, electric field strengths of about 5 to about 25 kV/cm, about 7 to about 23 kV/cm, about 10 to about 20 kV/cm, about 12 to about 18 kV/cm, or the like may be applied for about 10-1000 ns, about 25-750 ns, about 50-500 ns, about 75-250 ns, or the like.

Optionally, the eTE may be increased by controlling the number of pulses applied to each cell passing through the pulsing region. The number of pulses applied to each cell is inversely proportional to the flow rate of the cells because the residence time of the cell within an applied electric field is inversely proportional to the flow rate. As such, when the flow rate is increased, the cell residence time within the applied electric field decreases, and the number of pulses applied to each cell decreases. The flow rate may be controlled to control the cell velocity (by increasing or decreasing) such that the electric field strength level experienced by a cell is enough to promote electroporation (i.e., optimize eTE) while preventing cell death/electrolysis. In various embodiments, the flow rate may be differentially controlled at various positions within the microfluidic channel 101 (using, for example, valves, flow pumps, different diameters of the microfluidic channel, or the like).

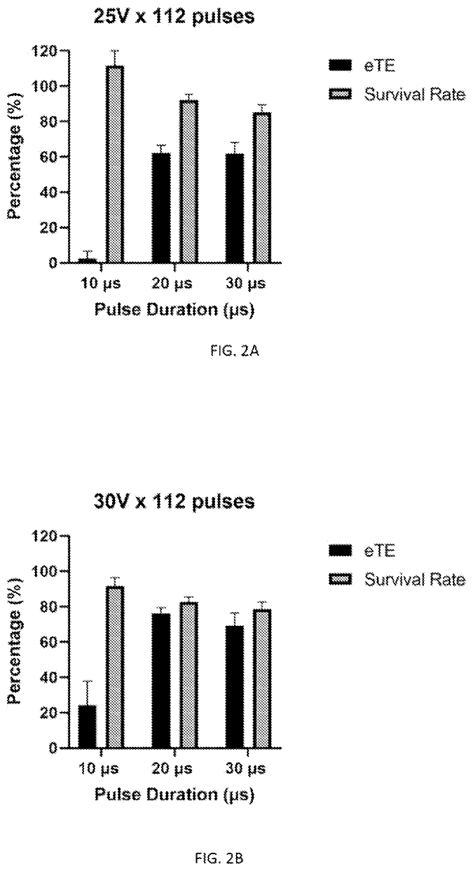

FIGS. 2A and 2B illustrate the effect of increasing the pulse duration eTE and survival rate (SR). In FIG. 2A, the treated cells experienced 112 pulses with an amplitude of 25 V (E=1.35 kV/cm) and duration of 10 μs, 20 μs or 30 μs. In FIG. 2B, the treated cells experienced 112 pulses with an amplitude of 30 V (E=1.62 kV/cm) and duration of 10 μs, 20 us or 30 μs. Survival rate was evaluated through the use of resazurin-based cell viability reagent (Presto Blue HS), which uses the reducing power of living cells to convert resazurin to fluorescent resorufin. As shown in FIGS. 2A and 2B, increasing the pulse duration from 10 μs to 20 μs dramatically increased eTE with some decrease in SR, while increasing the pulse duration from 20 μs to 30 μs had negligible effect on eTE and SR.

In various embodiments, the pulse duration may be about 5 μs to about 20 ms for an electric field strength of about 0.2-2.0 kV/cm. In such embodiments, the number of pulses may be about 1-250. In various other embodiments, the pulse duration may be about 50-1000 ns for an electric field strength of about 1-20 kV/cm. In such embodiments, the number of pulses may be about 1-2000.

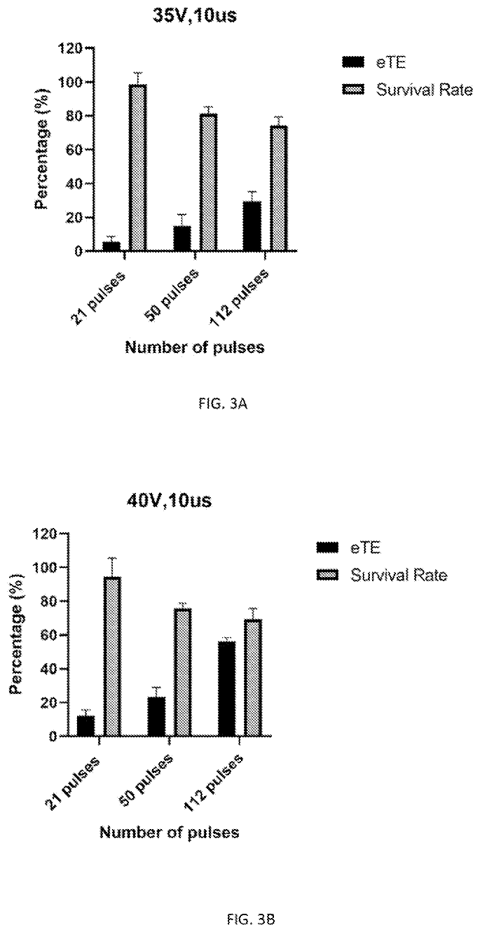

FIGS. 3A and 3B illustrate the effect of increasing the number of pulses on eTE and SR. In FIG. 3A, the treated cells experienced 21, 50 or 112 pulses with duration of 10 μs and pulse amplitude of 35V (E=1.89 kV/cm). In FIG. 3B, the treated cells experienced 21, 50 or 112 pulses with duration of 10 μs and pulse amplitude of 40V (E=2.16 kV/cm). Survival rate was evaluated through the use of resazurin-based cell viability reagent (Presto Blue HS). As shown in FIGS. 3A and 3B, eTE increased with an increase in number of pulses while the SR decreased.

Additionally and/or optionally, the eTE may also be improved by applying a first high field (“HV”) signal (i.e., a permeabilization signal) followed by a second low field (“LV”) signal (i.e., a delivery signal) using two spatially distant pair of electrodes (114(a)-(b) and 114(c)-(d) in the pulsing regions 104(a) and 104(b)) as shown in FIG. 1B.

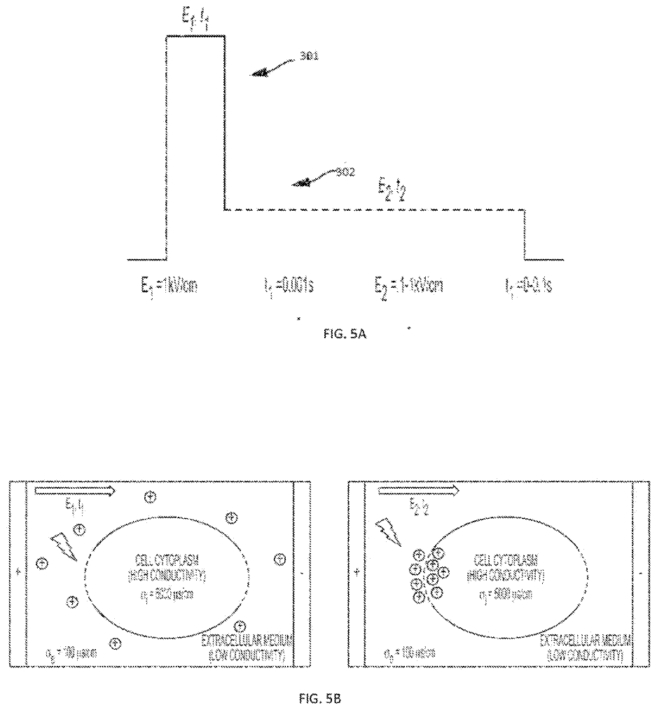

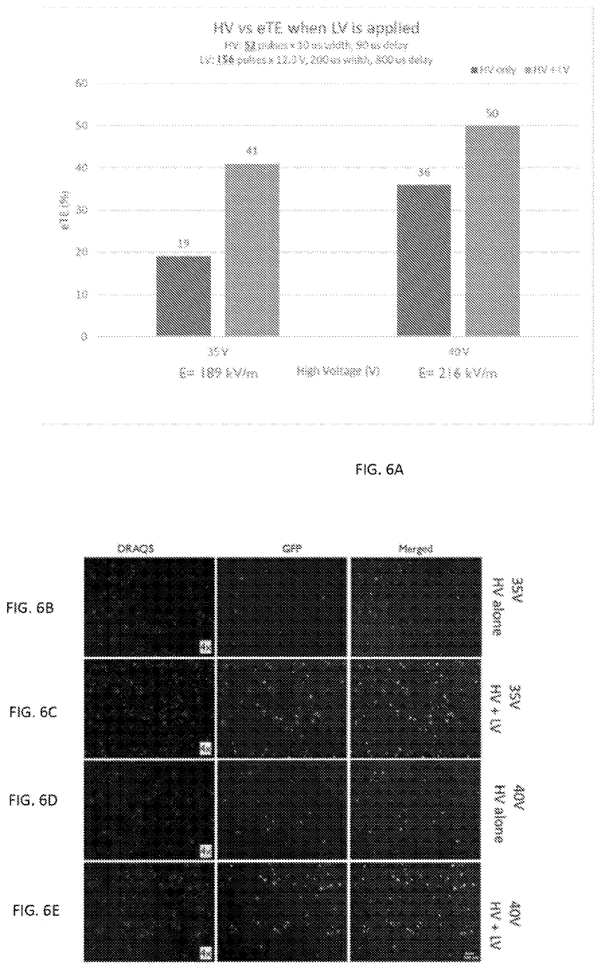

FIGS. 5A and 5B show the permeabilization signal and delivery signal as described above: a first signal 301 for permeabilization, followed in time by a second signal 302 for molecular delivery. The application of the first HV signal is in general a necessary condition for membrane permeabilization, to overcome the critical threshold of the transmembrane potential. On the other hand, once permeabilization is achieved, a LV signal can be employed to deliver the small and big molecules effectively while simultaneously decreasing damage due to field exposure. Hence, the two-signal electroporation system may be uniquely designed with the first signal being high amplitude, short duration (using electrodes 114(a)-(b)) which serves to permeabilize the cell membrane without irreversibly damaging the cells, and second signal being longer in duration and lower in amplitude (using electrodes 114(c)-(d)) serving to retain membrane pore opening and to electrophoretically drive molecules into cells. In certain embodiments, the flow rate of the cell as it passes between electrodes 114(a)-(b) (HV signal region) is different from the flow rate of the cell as it passes between electrodes 114(c)-(d) (HV signal region). FIG. 6A is a bar graph illustrating the change in eTE upon application of a single electroporation pulse compared to when two spatially separated signals (HV and LV) are applied at different voltage strengths. FIGS. 6B-6E illustrates a GFP expression in HEK293 cells when the HEK293 cells were incubated at 37 °C for 24 hours following micro-electroporation experiments. All cells were stained with DRAQ5 (red). The cells were treated as follows: FIG. 6B: 52×189 kV/m: 10 μs HV pulses alone; (FIG. 6C) 52×189 kV/m: 10 μs HV pulses followed by 156×66 kV/m: 200 μs LV pulses; (FIG. 6D) 52×216 kV/m: 10 μs HV pulses alone; (FIG. 6E) 52×216 kV/m: 10 μs HV pulses followed by 156×66 kV/m: 200 μs LV pulses.

In an embodiment, the HV signal is designed with the permeabilization signal high in amplitude (e.g., >1 kV/cm) but short in duration (e.g., <1 ms) to permeabilize the cell membrane; and the delivery signal lower in amplitude (<0.6 kV/cm) but longer in duration which serves to retain the opening of the pores from the first signal and electrophoretically transport molecules into the cell.

For a given, device design, other parameters that may be controlled to improve eTE without causing cell death and electrolysis may include, without limitation, voltage, frequency, duration, pulse amplitude, duty cycle, pulse type, pulse width, or the like. With a DC pulse train, there are several interrelated parameters that can potentially affect the delivery efficiency during electroporation, which may be altered to achieve the desired permeabilization: electric field amplitude, pulse duration, pulse train frequency, duty cycle and number of cycles. For example, a 50 kHz pulse train has a 20 μs pulse period, and with a 50% duty cycle, each pulse is 10 μs long. To obtain a 10 ms total pulse application, 1000 cycles are applied for 20 ms at 50 KHz frequency. The duty cycle controls the amount of rest period following each pulse. Any of the above parameters may be altered to achieve the desired premebilization level without causing electrolysis of the cell. For example, in an embodiment, the duty cycle can be tuned to increase the pulse width improving delivery time at the cost of electrolysis with the pulse train becoming more like a single DC pulse, or decreased to reduce delivery time while increasing the number of pulses to minimize electrolysis.

In some embodiments, the HV and/or the LV signals may be a DC pulse waveform, a series of DC pulse waveforms, an AC pulse waveform (e.g., sine waves), or combinations thereof. In certain implementations, the electrode pair(s) in the pulsing region 104 may be designed to further improve eTE by, for example, changing the geometry. For example, interdigitated electrodes (IDEs), triangle electrodes, or the like may be used to generate the electroporation signal(s). For example, as shown in FIGS. 1A and 1B, the electrode trace in a pulsing region may include a terminal region (adjacent the microfluidic channel) that is triangular in shape in order to improve efficiency and/or to prevent breakage of a thinner cross section electrical trace. Other shapes to increase the cross-section (such as square) are within the scope of this disclosure.

In other examples, interdigitated electrodes may be used. Interdigitated electrodes (IDEs) are fabricated through the process of combining two separately addressable electrode arrays, such that the resulting electrode structure is infused in a zipper-like or comb-shaped arrangement. An example device 170 including interdigitated electrodes 174(a) and 174(b) in the pulsing region 174 is illustrated in FIG. 1C. Optionally, the sensing regions 172 and/or 176 may also include interdigitated electrodes. Use of IDEs may improve throughput of the device because IDEs span across a longer length region of the microfluidic channel, obviating the need to hydrodynamically focusing the cells through the pulsing region between a pair of pulsing electrodes. For example, a throughput of electroporating about 1000-100,000 cells/second may be achieved (i.e., throughput required to address manufacturing needs for cell therapy applications or other applications).

The spacing may be controlled in such IDEs to allow high field strength pulses (by decreasing the spacing) at lower voltages to avoid solution electrolysis. For example, the spacing or gap between the electrodes may be decreased such that less voltage will be required to stimulate equal electric field amplitudes—this may be done using IDE electrodes allowing for lower voltages for the same eTE(<1.2V). IDEs with 20 μm electrode width−20 μm spacing and 1 Volt DC as stimulation induces an electric field with 50 kV/m magnitude, whereas 15 μm electrode width−15 μm spacing and just 1 Volt DC as stimulation induces an electric field with 66 kV/m magnitude. Table 3 illustrates the voltages at which electrolysis is observed for different voltages and buffers for the IDEs 20 μm electrode width−20 μm spacing and 15 μm electrode width−15 μm spacing:

TABLE 3

| | | | Voltage | Electric Field |
|---|---|---|---|---|
| Device | Buffer | Electrodes | Threshold (V) | (kV/M) |
| 15 × 15 | PBS | HV | 1.7 | 113 |
| | | LV | 2.5 | 166 |
| | CM | HV | 1.6 | 106 |
| | | LV | 2.4 | 160 |
| 20 × 20 | PBS | HV | 1.7 | 85 |
| | | LV | 1.5 | 75 |
| | CM | HV | 1.6 | 80 |
| | | LV | 1.5 | 75 |

These results suggest that electric fields with magnitude up to ~160 kV/m can be achieved with 15×15 IDEs without electrolysis.

Optionally, in some embodiments, as shown in FIG. 1B, a second sensing region 106 (using a pair of electrodes across the microfluidic channel 101 and/or three electrodes, as described below) may be included downstream of the pulsing region 104 in order to monitor the permeabilization state of the detected cell via impedance monitoring.

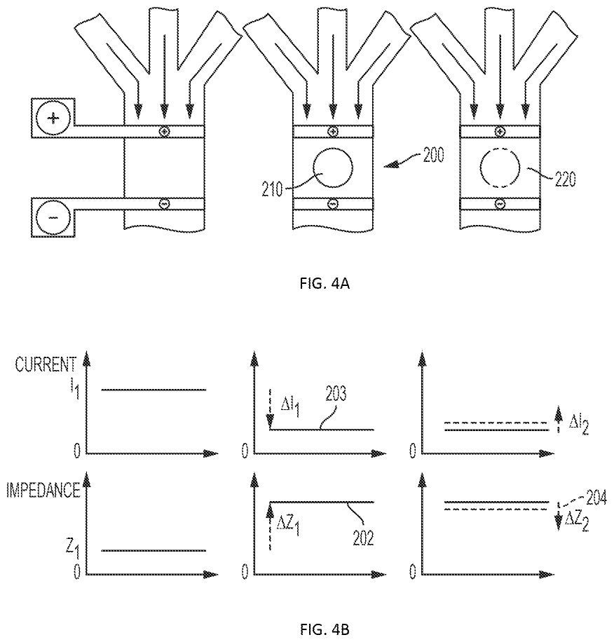

FIG. 4A and FIG. 4B illustrates the change in overall channel impedance as a cell moves through a microfluidic channel. An alternating current detection signal is applied across the microfluidic channel (e.g., in the first sensing region 102), and as the cell 210 is drawn through the microfluidic channel 200, it displaces the surrounding electrolyte causing a brief increase in electrical impedance under alternative current-based sensor detection 202 of the liquid (resembling resistive pulse), which is monitored through changes in electric current 203 across the channel, according to Ohm's law. This allows for enumeration of cells through the number of resistive pulses and sizing information based on the magnitude of the pulse, which in turn depends on the volume of displaced electrolyte. The increase in impedance occurs because the non-conducting, lipid bi-layer that comprises the cell membrane is intact and prevents open communication of the intracellular and intercellular solutions. Moreover, as discussed above, the cell type may also be determined based on the change in impedance in this sensing region. However, once permeabilized 220, the cell becomes more conductive, and the impedance drops 204 (and the electric current increases). The change in impedance may be detected in, for example, the second sensing region 106 to provide a signature of the permeabilization state of the cell as shown in FIGS. 4A and 4B, and various parameters of the pulsing region (e.g., voltage, frequency, duration, pulse amplitude, duty cycle, pulse type, number of pulses (by controlling velocity), pulse width, separating HV and LV pulsing regions, etc.) may be dynamically adjusted to switch to a well-tolerated customized field for the specific cell type and size to maximize delivery, as described herein (e.g., via a controller).

The electric field strength and duration may be tailored with respect to the target molecule to enhance overall performance by, for example, changing parameters such as the voltage, frequency, duration, pulse amplitude, duty cycle, pulse type, pulse width, number of pulses, separating HV and LV pulsing regions, or the like. In some embodiments, the applied field strength of the electroporation signal may range from 0.2 to 20 kV/cm with a duration between about 0.01 to about 30 ms. For example, in an embodiment, for any chosen cell type that undergo such electroporation, the initial output amplitude and duration information of the electroporation signal (or the HV and LV signals) may be chosen based on set values known in literature and may be inputted by the operator prior to system execution. In some embodiments, the parameters for common cell types may be stored in the system. In other embodiments, dependent upon the sensory sweep of the cell membrane impedance information during the application electroporation signal(s)—as discussed above—the central control algorithm retains the ability to modify (e.g., change parameters) the electroporation signal(s) based on the continuous tracking/sweeping of the cell membrane state in order to preserve cell viability. Continuous tracking of the cell membrane state may also provide information regarding the cell viability, and the second signal may be terminated either based on the cell viability close to reaching a point of irreversible damage (threshold determined through pre-calibration) or saturation of the delivered materials (threshold determined through pre-calibration). In an embodiment, the electroporation system may be designed to operate at a microscale level, and the electroporation signal(s) may be “chopped” into trains of DC pulses at adjustable frequencies (1 HZ-1 GHz, 0-100% duty cycles) with the appropriate amplitude adjustment to meet the permeabilization requirement, in order to measure the cell membrane permeabilization response during electroporation without the generation of electrolysis. In an embodiment, a database may be created comprising experimental characterization and/or computations modeling of the electroporation signal based on cell type, structure, buffer characteristics, microfluidic channel characteristics, etc. For example, a wide range of pulsing conditions may be tested on a cell population level and are analyzed to determine the optimal pulse features for a particular cell type's required permeabilization threshold and associated parameters, which may be stored in a database for future use. As discussed below the signal may then be designed based on the detected cell and other properties using the database and/or computations modeling based on the database.

In some example embodiments, the dimensions of the device such as, without limitation, the gap between electrode pairs in the first pulsing region (X1) and/or the second pulsing region (X2), distance (Y) between the first pulsing region 174(a) and the second pulsing region 174(b), length (L1) of the electrodes in the first pulsing region (i.e., electrodes 114(c) and 114(d), length (L2) of the electrodes in the second pulsing region (i.e., electrodes 114(a) and 114(b)), or the like; may be optimized to achieve a desired electroporation and throughput of the cells (based on, for example, residence time between electrodes and electric field strength). For example, the length L1 and/or L2 may be optimized to achieve a desired amount of time the cells will experience a given electric field (which may depend upon cell velocity and the lengths L1 and/or L2). The distances X1 and/or X2 may be optimized based on a desired electric field strength (the distance being inversely proportional to the respective distances). The distance Y may be optimized based on experimental or real-time data relating to how the length of time between pulses the first pulsing region 174(a) and the second pulsing region 174(b) affects eTE and cell viability.

In various embodiments, longer electrode lengths may be used to provide a longer residence time of a cell between the electrodes. Therefore, a faster flowrate/cell velocity can be used to increase throughput (the residence time the cell is between the electrode is the electrode width, L, divided by the cell velocity (U_{cell}), so t=L/U_{cell})). In other words, increasing L proportionally increases the perfusion rate which would process a greater number of cells in the device over the same time period increasing throughput.

In various embodiments, X1 and X2 may be about 25-200 μm, about 50-175 μm, about 75-150 μm, about 100-125 μm, or the like. In certain embodiments, the distance Y may be about 5-1000 μm, about 50-900 μm, about 100-800 μm, about 150-700 μm, about 200-600 μm, about 250-500 μm, about 300-400 μm, or the like. In various embodiments, L1 may be about 20-100 μm, about 30-90 μm, about 40-80 μm, about 50-70 μm, or the like. In various embodiments, L2 may be about 100-1000 μm, about 200-900 μm, about 300-800 μm, about 400-700 μm, about 500-600 μm or the like.

In various embodiments, the first sensing region and the second sensing region may each include three electrodes (instead of two) to form inline impedance cytometers configured to quantify the impedance change between the upstream (pre-electroporation) and downstream (post electroporation) cell impedance (as shown in FIG. 1B). Specifically, each cytometer will be comprised of three electrodes, configured so that the impedance signal between electrode pairs B and C is subtracted from the signal between pairs A and B. A lock-in amplifier will be used to inject AC excitation signals into the sensor's center counting electrode. Relative impedance will be measured using the Wheatstone bridge circuit, by acquiring output voltages V1 and V2 across the resistors, respectively. The output voltages may then be fed to a differential amplifier that cancels the common mode noise in between V1 and V2. The output voltage of a differential amplifier may be provided to the lock-in amplifier to further remove any unwanted noise.

Different cell types may be electroporated using the system, as described herein. Examples may include, without limitation, 3T3 fibroblasts, human dermal fibroblasts (HDFs), and lymphoblastoid cells (LCLs). Cells may be prepared for electroporation using techniques known to those skilled in the art. For example, the 3T3 fibroblasts may be maintained in complete cell media and cultured to 80% confluency before being harvested for experiments. Prior to electroporation, the cells are trypsinized and resuspended in an electroporation buffer. The electroporation buffer is an iso-osmotic solution of 250 mM sucrose, 10 mM HEPES, and a selected concentration of MgCl₂ salt at a pH of 7.4 (and/or a mixture of Mg²⁺ and K⁺ ions). Other buffers such as 1×PBS or tissue culture media may be used for, for example, therapy applications using HDFs or T lymphocytes. The amount of MgCl₂ added (ranging from 0.4-11.2 mM) determines the final conductivity of that extracellular buffer solution (ranging from 100-2000 μS/cm). The osmolarity of the solution is adjusted to a cell compatible 310 mOsm/kg using an Advanced Osmometer 3D3 (Advanced Instrument, Norwood MA). Trypsinized cells are introduced into the middle inlet of a 3-inlet microdevice via syringe pump (Harvard Apparatus, Cambridge, MA). The 3-inlet approach hydrodynamically focuses the cells to a width of about 20-25 μm to ensure that the cells enter the center of the operating region of the device in single file.

The systems and methods described in this disclosure may be used to deliver a variety of molecules whose sizes span from hundreds of Daltons to hundreds of thousands of Daltons. Smaller molecules can be delivered efficiently while retaining high levels of cell survival. Larger molecules, on the other hand, may require higher field strengths or longer pulses to drive them into cells, at a cost of greater cell death. By using the “smart” electroporator system of the current disclosure, delivery may be maximized while preserving viability. Examples of molecules that may be delivered into a cell using the systems and methods described herein include, without limitation, small organic compounds, such as drugs and molecular probes, small strands of RNA that are typically used as interfering RNA (siRNA), mRNA, proteins, and plasmid DNA for direct transfection.

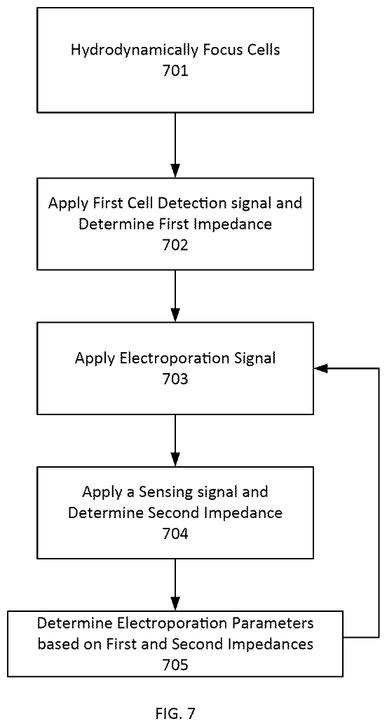

FIG. 7 provides a method for electroporation of single cells in a continuous flow using the smart electroporation device described above. As shown in FIG. 7, in step 701, cells in a continuous flow system are hydrodynamically focused such that a single cell is introduced into a defined upstream detection region. Concepts relating to hydrodynamic focusing are known to those skilled in the art. As discussed above, hydrodynamic focusing may not be required when the pulsing electrodes are IDEs.

In step 702, a cell detection signal is applied across the upstream detection area and the impedance is monitored. In some embodiments, the cell detection signal may be an AC detection waveform obtained either from simulation models or known literature. The AC detection waveform may be used to monitor the presence or absence of a cell within the detection area based on a change in impedance. Detection of a cell may trigger the smart electroporation system for signal application.

In step 703, an electroporation signal is applied across the pulsing region. As discussed above, an electroporation signal may include a single pulsing signal such as a short-duration, high frequency DC pulse for permeabilization followed by a long-duration, low frequency DC signal for delivery into the cell, as described above. Optionally, the electroporation signal can include a HV pulsing signal applied in a first upstream region followed by a second LV pulsing signal applied in a downstream region of the microfluidic channel.

Optionally, permeabilization status of the cell following may be determined by applying a sensing signal downstream of the electroporation region (704). For example, changes in the cell impedance following electroporation may be detected by monitoring the impedance of a permeabilized cell as it passes through a second sensing region. In an embodiment, the parameters of the electroporation signal for 703 as well as the flow of cells through the pulsing region may be controlled based on a difference between the impedance detected using the upstream detection signal and the downstream sensing signal (705). As such, the electroporation signal(s) in the pulsing region(s) may be dynamically controlled using a feedback control that continuously monitors the impedance changes pre and post electroporation. In such embodiments, the detected impedance in the first sensing region may be compared to the detected impedance in the second sensing region (as discussed above) to control various parameters of the electroporation signal and/or the flow of cells through the pulsing region.

In various embodiments, the difference in upstream and downstream impedance (i.e., before and after permeabilization) of the cell may be correlated to experimental data to determine electroporation parameters. The experimental data may be received from using the electroporation system on immobilized cells (for a particular cell type), where the experimental data is optimized to determine the optimal electroporation parameters and cell velocity for various cell types. In various embodiments, the electroporation signal may be experimentally predesigned based on the pulse characteristics of the first and second detection signals (and corresponding impedance differences), cell type, cell size, buffer characteristics, and other properties and desired eTE and/or survival rate (as discussed above).

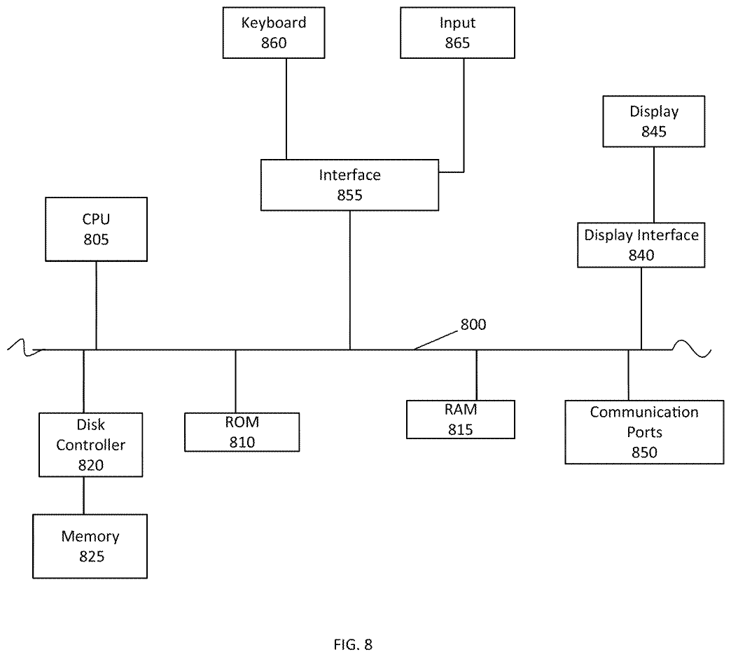

FIG. 8 depicts an example of internal hardware that may be used to contain or implement the various computer processes and systems as discussed above. For example, the smart electroporation discussed above may include hardware such as that illustrated in FIG. 8. An electrical bus 800 serves as an information highway interconnecting the other illustrated components of the hardware. CPU 805 is a central processing unit of the system, performing calculations and logic operations required to execute a program. CPU 805, alone or in conjunction with one or more of the other elements, is a processing device, computing device or processor as such terms are used within this disclosure. A CPU or “processor” is a component of an electronic device that executes programming instructions. The term “processor” may refer to either a single processor or to multiple processors that together implement various steps of a process. Unless the context specifically states that a single processor is required or that multiple processors are required, the term “processor” includes both the singular and plural embodiments. Read only memory (ROM) 810 and random access memory (RAM) 815 constitute examples of memory devices. The term “memory device” and similar terms include single device embodiments, multiple devices that together store programming or data, or individual sectors of such devices.

A controller 820 interfaces with one or more optional memory devices 825 that service as date storage facilities to the system bus 800. These memory devices 825 may include, for example, an external or internal disk drive, a hard drive, flash memory, a USB drive or another type of device that serves as a data storage facility. As indicated previously, these various drives and controllers are optional devices. Additionally, the memory devices 825 may be configured to include individual files for storing any software modules or instructions, auxiliary data, incident data, common files for storing groups of contingency tables and/or regression models, or one or more databases for storing the information as discussed above.

Program instructions, software or interactive modules for performing any of the functional steps associated with the processes as described above may be stored in the ROM 810 and/or the RAM 815. Optionally, the program instructions may be stored on a non-transitory, computer readable medium such as a compact disk, a digital disk, flash memory, a memory card, a USB drive, an optical disc storage medium, and/or other recording medium.

An optional display interface 840 may permit information from the bus 800 to be displayed on the display 845 in audio, visual, graphic or alphanumeric format. Communication with external devices may occur using various communication ports 850. A communication port 850 may be attached to a communications network, such as the Internet, a local area network or a cellular telephone data network.

The hardware may also include an interface 855 which allows for receipt of data from input devices such as an imaging sensor 860 of a scanner or other input device 865 such as a keyboard, a mouse, a joystick, a touchscreen, a remote control, a pointing device, a video input device and/or an audio input device.

The above-disclosed features and functions, as well as alternatives, may be combined into many other different systems or applications. Various presently unforeseen or unanticipated alternatives, modifications, variations or improvements may be made by those skilled in the art, each of which is also intended to be encompassed by the disclosed embodiments.

What is claimed is:

1. A system for electroporating a biological cell, the system comprising:
a microfluidic channel adapted to receive a flow of a plurality of biological cells in a buffer solution, wherein the microfluidic channel comprises a detection region and a pulsing region;
a first pair of electrodes adapted to apply a first electrical field across the detection region; and
a second pair of electrodes adapted to apply a second electric field across the pulsing region.

2. The system of claim 1, further comprising a second detection region downstream of the pulsing region.

3. The system of claim 2, further comprising a controller unit, wherein the controller unit is adapted to control parameters of the second electric field based on a first impedance detected in the detection region and a second impedance detected in the second detection region.

4. The system of claim 1, further comprising a third pair of electrodes downstream of the second pair of electrodes adapted to apply a third electric field across the pulsing region.

5. The system of claim 4, wherein the second electric field has a strength that is higher than that of the third electric field.

6. The system of claim 4, wherein the second electric field has a shorter duration than the third electric field.

7. The system of claim 4, wherein the second pair of electrodes or the third pair of electrodes comprise interdigitated electrodes.

8. The system of claim 1, wherein the first electrical field is configured to detect presence of a cell between the first pair of electrodes.

9. The system of claim 1, wherein the second electrical field is configured to electroporate the cell between the second pair of electrodes.

10. The system of claim 1, further comprising a plurality of inlets configured to hydrodynamically focus the plurality of biological cells in a single cell flow within the microfluidic channel.

11. A method for electroporating a biological cell, the method comprising:
receiving, in a microfluidic channel, a flow of a plurality of biological cells in a buffer solution;
applying a first electrical field across a detection region of the microfluidic channel, the first electric field being configured to detect presence of a cell; and
applying a second electric field across a pulsing region of the microfluidic channel, the second electric field being configured to electroporate the cell.

12. The method of claim 12, further comprising applying a third electric field in a second detection region of the microfluidic channel, the second detection region being downstream of the pulsing region.

13. The method claim 12, further comprising controlling parameters of the second electric field based on a first impedance detected in the detection region and a second impedance detected in the second detection region.

14. The method claim 11, further comprising applying a third electric field across the pulsing region.

15. The method claim 14, wherein the second electric field has a strength that is higher than that of the third electric field.

16. The method claim 14, wherein the second electric field has a shorter duration than the third electric field.

17. The method claim 14, wherein the second electric field is configured to cause permeabilization of the cell.

18. The method claim 14, wherein the third electric field is configured to cause delivery of a molecule in the cell.

19. The method claim 11, further comprising detecting presence of the cell in response to detecting a rise in impedance in the detection region.

20. The system of claim 1, further comprising hydrodynamically focusing the plurality of biological cells in a single cell flow within the microfluidic channel.
